(19) United States
(12) Patent Application Publication
(10) Pub. No.: US 2006/0062774 A1
(43) Pub. Date: Mar. 23, 2006

(54) Compositions for maintaining and restoring normal urogenital flora

(75) Inventors: Catherine Cornick Davis, Sharonville, OH (US); Thomas Ward Osborn III, Clifton, OH (US); Kenneth Wallace Miller, Liberty Twp, OH (US); Bruce Elliott Jones, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(73) Assignee: The Procter & Gamble Company

(21) Appl. No.: 11/205,984

(22) Filed: Aug. 17, 2005

Related U.S. Application Data
(60) Provisional application No. 60/612,398, filed on Sep. 21, 2004.

(51) Int. Cl. A61K 35/74

(52) U.S. Cl. 424/93.45

(57) ABSTRACT

A composition and methods for maintaining and restoring normal urogenital flora in humans. The composition includes one or more bacteria selected from the group consisting of Lactobacillus iners, all clones with at least 97% sequence similarity to Lactobacillus iners, Atopobium spp, and all clones with at least 90% sequence similarity to Atopobium spp as determined by sequences from 16S rRNA genes. The method for maintaining includes administering a safe and effective amount of the composition. The method for restoring includes administering a safe and effective amount of an antibiotic and subsequently administering a safe and effective amount of the composition.

### CROSS REFERENCE TO RELATED APPLICATION

This application claims the benefit of U.S. Provisional Application No. 60/612,398, filed Sep. 21, 2004.

### FIELD OF THE INVENTION

This invention relates to bacterial compositions and methods for maintaining and restoring normal indigenous urogenital flora in a human. These compositions and methods may be employed to treat or prevent urogenital diseases and infections.

### BACKGROUND OF THE INVENTION

It is well known that indigenous, non-pathogenic bacteria predominate on epithelial cells and associated mucus in the healthy state, and that pathogenic organisms predominate in the stages leading to and during infections. The possibility that indigenous bacteria have a role in preventing infection has been postulated for many years, but few studies have been carried out to identify specific bacteria and their properties required for such an effect.

Bacteria are the predominate type of microorganisms present in the vagina, and most women harbor about 109 bacteria per gram of vaginal secretions. The bacterial flora of the vagina is comprised of both aerobic and anaerobic bacteria. The more commonly isolated bacteria include Lactobacillus species, Corynebacteria species, Gardnerella vaginalis, Staphylococcus species, Peptococcus species, aerobic and anaerobic streptococcal species, and Bacteroides/Prevotella species. Other microorganisms that have been isolated from the vagina on occasion include yeast (Candida albicans), protozoa (Trichomonas vaginalis), mycoplasma (Mycoplasma hominis), chlamydia (Chlamydia trachomatis), and viruses (Herpes simplex). These latter organisms are generally associated with vaginitis or sexually transmitted diseases, although they may be present in low numbers without causing symptoms.

Physiological, social and idiosyncratic factors affect the quantity and species of bacteria present in the vagina. Physiological factors include age, days of the menstrual cycle, and pregnancy. For example, vaginal flora present in the vagina throughout the menstrual cycle can include species of Lactobacillus, Corynebacteria, Ureaplasma, and Mycoplasma. Social and idiosyncratic factors include presence and method of birth control, sexual practices, systemic disease (e.g., diabetes), and medication.

Bacterial proteins and metabolic products produced in the vagina can affect other microorganisms and the human host. For example, generally the pH of the vagina between menstrual periods is mildly acidic, having a pH ranging from about 3.8 to about 4.5. This pH range is generally considered the most favorable condition for the maintenance of normal flora. At that pH, the vagina normally harbors the numerous species of microorganisms in a balanced ecology, playing a beneficial role in providing protection and resistance to infection and making the vagina inhospitable to some species of bacteria such as Staphylococcus aureus. The low pH is a consequence of the growth of lactobacilli and their production of acidic products. Microorganisms in the vagina can also produce antimicrobial compounds such as hydrogen peroxide and bacteriocins which attack and eliminate other bacterial species. One example is the lactocins, bacteriocin-like products of lactobacilli directed against other species of Lactobacillus. Some microbial products may affect the human host. For example, S. aureus can produce and excrete into its environment a variety of exoproteins including hemolysins, enterotoxins, toxic shock syndrome toxin-1 (“TSST-1”), and enzymes such as protease and lipase.

Vaginal menstrual toxic shock syndrome (TSS) is a rare syndrome characterized by rapid onset of high fever, vomiting, diarrhea, and rash followed by a rapid drop in blood pressure and vital organ failure. TSS is associated with the presence of S. aureus bacteria and one or more exotoxins which are produced by the bacteria. The exotoxins associated with menstrual TSS include but may not be limited to staphylococci: Enterotoxin A, Enterotoxin B, Enterotoxin C, and TSST-1. Using traditional culture based techniques, S. aureus has been identified in the vagina of approximately 16% of healthy women of menstrual age. However, recent clinical studies using DNA based techniques have shown this number may be much higher. It has been found that approximately 10% of the S. aureus isolated from the vagina are capable of producing TSST-1. TSS is not caused by the bacteria per se but rather by the toxic effects of the associated exotoxin which can pass from the vagina and other internal body cavities into the blood stream.

TSS has been associated with the use of absorbent pads within the vagina which may promote the growth of bacteria and the production of exotoxin in their vicinity. The syndrome has been observed with surgical dressings, and is also associated with the use of catamenial tampons. The syndrome appears to occur with elevated frequency in association with those absorbent pads which are characterized by high levels of absorbency and which accordingly are left inside the body for extended periods. While a preferred approach for reducing the risk of TSS when using absorbent pads is proper use and frequent changes of new pads for used ones, various other approaches have been proposed by the art for reducing the risk of TSS associated with an internal absorbent pad. One approach is the incorporation of antimicrobial or bacteriocidal agents into the absorbent pad such as the use of iodine bacteriocides in tampons and catamenial sponges. Such an approach is not always suitable for use in the catamenial product, however, because a bacteriocide which is active against S. aureus can adversely affect other beneficial bacteria which make up the vaginal flora, thereby upsetting the healthy balance discussed above. Another related method describes the use of catamenial tampons comprising substances such as organic acids which will maintain a pH of about 4.5 to about 2.5 in the fluids absorbed during the use of the tampon such that the growth of pathogenic bacteria is inhibited.

Other approaches are directed to inactivation of the TSST-1 such as the administration of L-ascorbic acid for the detoxification of the S. aureus toxins, (TSST-1). While this method does not ascribe a mechanism for the effectiveness of ascorbic acid at neutralizing TSST-1, it observes that L-ascorbic acid is known to be a reducing agent and strong antioxidant and that it may operate to inactivate bacterial toxins by reducing disulfide bonds within the toxins.

Bacterial vaginosis is the consequence of a bacterial interaction responsible of substitution at the vagina region of a great number of anaerobic bacteria including Bacteroides, Peptostreptococcus, Peptococcus, Mobiluncus, Gardnerella vaginalis, for lactobacilli. This syndrome is characterized by a smelly although not particularly irritatating secretion. There is only a slight itching and dyspareunia is exceptionally uncommon. Bacterial vaginosis is commonly diagnosed among women that have vaginal trouble, although 50% of women responding to diagnosis criteria for bacterial vaginosis are asymptomatic. Bacterial vaginosis is associated with sexual activity and increases as the number of sexual partners increases; however this affection is not considered as an exclusively sexual-transmission disease.

Although bacterial vaginosis is slightly troublesome, it may predispose to development of more serious infections of the genital system, in particular during pregnancy. A diagnosis of bacterial vaginosis is given when three of the following four symptoms are encountered: (1) a homogeneous non-inflammatory vaginal secretion, adhering to vagina walls; (2) a vaginal fluid having pH greater than 4.5; (3) indicator cells “Clue cells”; and (4) a fishy amine smell of the vaginal secretion before or after addition of 10% potassium hydroxide (Whiff test).

Vaginitis is characterized by an abnormal vaginal secretion, local irritation and vulvar itch. The above are the symptoms of a local infection due to infectious agents such as Trichomonas vaginalis, or Candida spp. (above all Candida albicans). Vaginosis and symptomatic vaginitis are associated with diabetes, parathyroid insufficiency, altered defenses of the host organism, corticosteroid treatment, broad-spectrum antibiotic treatment, oral contraceptive drugs and pregnancy. Itch and secretion are the main symptoms of vaginitis due to Candida spp. Occasionally dyspareunia occurs. Vulvar erythema and vulvovaginal thrush can be noticed. For a diagnosis of vaginitis due to Candida spp. the presence of local symptoms is required because usually women can have Candida spp. in the vagina without showing any trouble.

The results of recent studies have encouraged acknowledgment of the important role accomplished by lactobacilli in keeping a normal bacterial equilibrium within the dynamic ecosystems, such as vagina, and for prevention of genital infections caused by pathogenic organisms. It is well known that many lactobacilli colonize the vagina of healthy adult women. They propagate using as an energy source the glycogen stored in the vaginal mucosae and compete with the pathogenic agents from the latter, so as to keep the vaginal lumen defended from the attack of other bacteria. Sulfonamides, antibiotics, and other and disinfectants such as iodine have been mainly used for treatment of the above mentioned diseases (non-specific vaginitis, vaginosis and vulvar itch, for example). In particular, in the most recent years, with the development of many antibiotics, often antibiotic preparations have been administered to patients. Lactobacilli present in the vagina can succumb to administration of these antibiotics. Unfortunately, pathogenic bacteria (staphylococci, for example) can acquire a resistance to antibiotics and thus make it difficult to treat these diseases by administering antibiotics.

In U.S. Pat. No. 5,176,911 use of lactobacilli has been proposed in gynecology for treatment of vaginal infections. In U.S. Pat. No. 6,277,370, the use of Lactobacillus casei, Lactobacillus gasseri, Lactobacillus fermentum, Lactobacillus casei subs. pseudoplantarum, Lactobacillus crispatus, and Lactobacillius casei for treatment of vaginal infections. In U.S. Pat. No. 6,180,100, the use of Lactobacillus casei, Lactobacillus acidophilus, Lactobacillus plantarum, and Lactobacillus jensenii for treatment of urinary tract infections.

This invention relates to bacterial compositions and methods for maintaining and restoring normal indigenous urogenital flora in a human. These compositions and methods may be employed to treat or prevent urogenital diseases and infections.

### SUMMARY OF THE INVENTION

The present invention relates to a composition and methods for maintaining and restoring normal indigenous urogenital flora in a human. The composition comprises one or more bacteria selected from the group consisting of Lactobacillus iners, all clones also referred to as isolates with at least 97% sequence similarity to Lactobacillus iners, Atopobium spp, and all clones with at least 90% sequence similarity to Atopobium spp as determined by sequences from 16S rRNA genes. The method for maintaining comprises administering one or more bacteria selected from the group consisting of Lactobacillus iners, all clones with at least 97% sequence similarity to Lactobacillus iners, Atopobium spp, and all clones with at least 90% sequence similarity to Atopobium spp as determined by sequences from 16S rRNA genes. The method for restoring comprises administering a safe and effective amount of an antibiotic and subsequently administering a safe and effective amount of a composition comprising one or more bacteria selected from the group consisting of Lactobacillus iners, all clones with at least 97% sequence similarity to Lactobacillus iners, Atopobium spp, and all clones with at least 90% sequence similarity to Atopobium spp as determined by sequences from 16S rRNA genes.

### DETAILED DESCRIPTION OF THE INVENTION

As used herein “applicator” refers to a device or implement that facilitates the insertion of a tampon, medicament, treatment device, visualization aid, or other into an external orifice of a human, such as the vagina, rectum, ear canal, nasal canal, or throat. Non-limiting specific examples of such include any known hygienically designed applicator that is capable of receiving a tampon may be used for insertion of a tampon, including the so-called telescoping, tube and plunger, and the compact applicators, an applicator for providing medicament to an area for prophylaxis or treatment of disease, a spectroscope containing a microcamera in the tip connected via fiber optics, a speculum of any design, a tongue depressor, a tube for examining the ear canal, a narrow hollow pipe for guiding surgical instruments, and the like.

As used herein, the term “deactivation” means to make less toxic or nontoxic.

As used herein, the term “density” is used with its common technical meaning with units of g/cm³ or g/cc. The density may refer specifically to that of a specific region or feature of the tampon as noted. The density will be measured, unless otherwise noted, but taking the weight divided by the geometric volume described by the shape. Unless noted, density refers to that of the overall structure and not the individual components, and will include in the measurement void volume of small pores and voids within the overall structure.

As used herein, the term “encapsulation” means the surrounding off or “caging” of a compound using a physical or chemical component.

As used herein, the term “inhibit” to prevent the normal growth of an organism or the activity of an enzyme or protein. As follows. “inhibitor” is any agent that prevents the normal growth of an organism or the activity of an enzyme or a protein.

The term “interlabial pad” refers to an absorbent product intended for the absorption of menstrual fluid or urine from the vaginal area by placement within the outer opening of the vagina. The interlabial pad comprises a liquid pervious topsheet, liquid impervious backsheet and an absorbent core disposed between the topsheet and the backsheet. Examples of such devices are described in U.S. Pat. No. 2,917,049 issued to Delaney on Dec. 15, 1959, U.S. Pat. No. 3,420,235 issued to Harmon on Jan. 7, 1969, U.S. Pat. No. 4,595,392 issued to Johnson, et al. on Jun. 17, 1986, and U.S. Pat. No. 5,484,429 issued to Vukos, et al. on Jan. 16, 1996. A commercially available interlabial device is the INSYNC Miniform interlabial pad which is marketed by A-Fem of Portland, Oreg. and described in U.S. Pat. Nos. 3,983,873 and 4,175,561 issued to Hirschman on Oct. 5, 1976 and Nov. 27, 1979, respectively.

The term “joined” or “attached,” as used herein, encompasses configurations in which a first element is directly secured to a second element by affixing the first element directly to the second element; configurations in which the first element is indirectly secured to the second element by affixing the first element to intermediate member(s) which in turn are affixed to the second element; and configurations in which the first element is integral with the second element; i.e., the first element is essentially part of the second element.

The term “overwrap” refers to the external surface of a disposable article such as a sanitary napkin, pantiliner, interlabial device, tampon, disposble diapers, and the like. In tampon embodiments, the overwrap typically comprises a fluid permeable layer that surrounds the absorbent tampon's absorbent structure and is the portion, which is direct contact with the vaginal lining during use.

As used herein, the terms “pantiliner,” and “sanitary napkin,” refers to absorbent articles worn external about the pudenal region for the absorption of fluid therefrom, to aid in wound healing, or for the delivery of active materials, such as medicaments, or moisture. Sanitary napkins typically comprise a liquid pervious topsheet, liquid impervious backsheet and an absorbent core disposed between the topsheet and the backsheet. The sanitary napkin, as well as each layer or component thereof can be described as having a “body facing” surface and a “garment facing” surface. Pantiliners and sanitary napkin may have side extensions commonly referred to as “wings,” designed to wrap the sides of the crotch region of the panties of the user of sanitary napkin that may be extension of the topsheet and/or the backsheet. Such devices are disclosed in U.S. Pat. No. 4,463,045 issued to Ahr et al., U.S. Pat. No. 4,556,146 issued to Swanson et al., U.S. Pat. No. 4,950,264 issued to Osborn III, et al. and U.S. Pat. No. 4,687,478 issued to Van Tillburg.

By “pharmaceutically-acceptable carrier” as used herein is meant one or more compatible solid or liquid filler diluents, or encapsulating substances. By “compatible” as used herein is meant that the components of the composition are capable of being commingled without interacting in a manner which would substantially decrease the pharmaceutical efficacy of the total composition under ordinary use situations. Some examples of substances which can serve as pharmaceutical carriers are sugars, such as lactose, glucose and sucrose; starches such as corn starch and potato starch; cellulose and its derivatives such as sodium carboxymethycellulose, ethylcellulose and cellulose acetates; powdered tragancanth; malt; gelatin; talc; stearic acids; magnesium stearate; calcium sulfate; vegetable oils, such as peanut oils, cotton seed oil, sesame oil, olive oil, corn oil and oil of theobroma; polyols such as propylene glycol, glycerine, sorbitol, manitol, and polyethylene glycol; agar; alginic acids; pyrogen-free water; isotonic saline; and phosphate buffer solution; skim milk powder; as well as other non-toxic compatible substances used in pharmaceutical formulations. Wetting agents and lubricants such as sodium lauryl sulfate, as well as colouring agents, flavouring agents, lubricants, excipients, tabletting agents, stabilizers, anti-oxidants such as ascorbic acid and vitamin E and preservatives, can also be present.

By “safe and effective amount” as used herein is meant a concentration high enough to significantly-positively modify the condition to be treated but low enough to avoid serious side effects (at a reasonable benefit/risk ratio), within the scope of sound medical judgment. A safe and effective amount of lactobacillus will vary with the particular condition being treated, the age and physical condition of the patient being treated, the severity of the condition, the duration of treatment, and the nature of concurrent therapy.

As used herein, a tampon has a “self-sustaining shape” when a tampon pledget has been compressed and/or shaped such that it assumes a general shape and size, which is vaginally insertable, absent external forces. It will be understood by one of skill in the art that this self-sustaining shape need not, and preferably does not persist during actual use of the tampon. That is, once the tampon is inserted and begins to acquire fluid, the tampon may begin to expand and may lose its self-sustaining form.

As used herein, the term “tampon,” refers to any type of absorbent structure that is inserted into the vaginal canal or other body cavities for the absorption of fluid therefrom, to aid in wound healing, or for the delivery of active materials, such as medicaments, or moisture. The tampon may be compressed into a generally cylindrical configuration in the radial direction, axially along the longitudinal axis or in both the radial and axial directions. While the tampon may be compressed into a substantially cylindrical configuration, other shapes are possible. These may include shapes having a cross section that may be described as rectangular, triangular, trapezoidal, semi-circular, hourglass, serpentine, or other suitable shapes. Tampons have an insertion end, withdrawal end, a length, a width, a longitudinal axis, a radial axis and an outer surface. The tampon's length can be measured from the insertion end to the withdrawal end along the longitudinal axis. A typical compressed tampon for human use is 30-60 mm in length. A tampon may be straight or non-linear in shape, such as curved along the longitudinal axis. A typical compressed tampon is 8-20 mm wide. The width of a tampon, unless otherwise stated in the specification, corresponds to the length across the largest cylindrical cross-section, along the length of the tampon.

The term “urogenital” as used herein, are intended to be synonymous and refer to the perineum, vulva, labial majora, all tissues enclosed by the labia majoria including the labia minora, clitoris, introitus, fourchette, hymenal remnants, the vestibule and all major (e.g. Bartholin's) and minor vestibular glands, all sebaceous glands, the urethra and periurethral glands (e.g. Skene's glands) and internal organs including the urethra, ureters, and bladder.

The term “cfu” as used herein, are intended to refer to its common technical meaning as number of microbial colony forming units.

The term “gastrointestinal” as used herein, are intended to be synonymous and refer to the oral cavity, esophagus, stomach, small intestines, large intestines, colon, anus and perianal region.

The term “nasal” as used herein, are intended to be synonymous and refer to the nose, sinus and connecting cavities.

The term “wipes” as used herein refers to a substrate used for the absorption of fluid from the body, to aid in wound healing, or for the delivery of active materials, such as medicaments, or moisture.

The present invention relates to a composition and method for maintaining and restoring normal indigenous urogenital flora in a human. The composition and method comprise one or more species of bacteria.

The composition for maintaining and restoring normal indigenous urogenital flora in a human is selected from Lactobacillus iners, all clones with at least 97% sequence similarity to Lactobacillus iners, Atopobium spp, and all clones with at least 90% sequence similarity to Atopobium spp. The degree of similarity is determined by sequence similarity of the 16S rRNA genes. Methods for determining the sequences and the degree of similarity are described by Pavlova S I et. al. in J. Appl. Microbiol. 202;202;92(3)451-9 and by Zhou et. al. Microbiology. 203 August; 150(pt 8):2565-73. The composition may comprises any species of Atopobium but Atopobium vaginae and all clones with at least 97% sequence similarity to Atopobium vaginae is typically used. The composition may further comprise Lactobacillus crispatus. The composition may also further comprise one or more species of bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus plantarum, Lactobacillus fermentum, Lactobacillus jensenii, Lactobacillus coleohominis, Lactobacillus vaginas, Anaerococcus spp., Clostridium spp., Dialister spp., Enterococcus faecalis, Finegoldia magna, Gardnerella vaginalis, Gemella palaticanis, Lachnospiraceae spp., Leptotrichia spp., Megasphaera spp., Streptococcus spp., Hydrogenophaga palleronii, Comamonas spp., Peptostreptococcus, spp., Aerococcus, spp., Veillonella, spp., Mycoplasma spp., Micromonas spp., and Gemella palaticanis.

The composition can comprise a safe and effective amount of one or more of the aforementioned bacteria with a pharmaceutically acceptable carrier.

This invention is not intended to be limited to any particular mode of application. Therefore oral, intravaginal, intraurethral or periurethral applications of the compositions can be used. The composition can be administered or applied in the form selected from the group consisting of a cream, paste, gum, a suppository, douche, mucoadehsive, liquid dental transport medium, moist wipe, microspheres, an ointments, an oral tablet, a liquid, a drink, a gel, and nasal spray.

One vehicle for delivery of beneficial bacteria may be microspheres comprised of poly (D.L-lactide-co-glycolide)(PLGA) and poly(D,L-lactide)(PLA) micropheres as described in Goodman, et al, Microsheres Under In Vitro Release Conditions, APPS PharmSCiTech, 2003: 4(4) article 50. Other methods for delivery or other mucoadhesives are described in U.S. Pat. No. 6,509,028 issued to Williams, et. al on Jan. 21, 2003. Another vehicle for delivery of a beneficial bacteria is an anaerobic dental transport medium available commercially from Anaerobe Systems, Morgan Hill, Calif.

Some forms of the composition may comprise one or more bacteria in a jelly base, preferably a K-Y jelly base. Another application involves the preparation of a freeze-dried capsule comprising the composition of the present invention. Effective dosages may range from 10³ to 10¹³ cfu per daily dose and more preferably from 10⁵ to 10¹⁰ cfu/ml per daily dose. Typically effective dosages are in the range of 10⁹ cfu/ml.

The treatment method may vary according to the individual condition of the subject. For example, one regimen involves the subject taking a continuous self administered dose one or more times a day. Another regimen involves the subject self administering a single dose at least once per week on an on-going basis. Yet another regiment involves the subject self administering one or more doses for a period of 1 to 120 days.

The method for maintaining comprises administering one or more bacteria selected from the group consisting of Lactobacillus iners and all clones with at least 97% sequence similarity to Lactobacillus iners, Atopobium spp, and all clones with at least 90% sequence similarity to Atopobium spp. The method for restoring comprises administering a safe and effective amount of an antibiotic and subsequently administering a safe and effective amount of a composition comprising one or more bacteria selected from the group consisting of Lactobacillus iners, all clones with at least 97% sequence similarity to Lactobacillus iners, Atopobium spp, and all clones with at least 90% sequence similarity to Atopobium spp. The degree of similarity is determined by sequence similarity of the 16S rRNA genes.

The antibiotic used in the method is selected from the group consisting of Penicillins, Cephalosporins, Clindamycin, Vancomycin, Tetracyclines, Chlorampheicol, Trimethoprim-Sulfamethoxazole, Erythromycins, Glycopeptides, Metronidazole, Sulfa drugs, Iodine and Chlorhexidine and mixtures thereof.

The composition used in the method may comprise a composition comprising Lactobacillus iners and Atopobium spp. The composition may include any species of Atopobium including Atopobium vaginae and all clones with at least 97% sequence similarity to Atopobium vaginae. The composition may further comprise Lactobacillus crispatus. The composition may comprise one or one species of bacteria selected from the group consisting of Lactobacillus casei, Lactobacillus gasseri, Lactobacillus fermentum, Lactobacillus amylolyticus, Lactobacillus acidophilus, Lactobacillus casei subs.pseudoplantarum, Lactobacillus brevis, Lactobacillus salivarius, Lactobacillus acidophilus, Lactobacillus. plantarum, Lactobacillus fermentum, Lactobacillus jensenii, Lactobacillus coleohominis, Lactobacillus vaginas, Anaerococcus spp., Clostridium spp., Dialister spp., Enterococcus facecalis, Finegoldia magna, Gardernella vagianlis, Gemella palaticanis, Lachnospiraceae spp, Leptotrichia spp, Meagsphaera spp., Streptococcus spp., Hydrogenophaga palleronil, Comamonas spp., Peptostreptococcus spp., Aerococcus, spp., Veillonella, spp, Mycoplasma spp., Micromonas spp., and Gemella palaticanis.

The method may comprise applying the composition directly to the urogenital region of a human with a device selecting from the group consisting of tampons, pantiliners, sanitary pad, interlabial pad, overwrap, wipes, and pessaries.

Although the present invention is not bound by any one theory or mode of operation, it is believed that, at least to some degree, that the inclusion of lactobacillus iners with other lactobacillus species provide the opportunity for the body to re-establish a healthy flora by reducing or excluding the population of pathogenic bacteria in the vagina, urinary tract, gastrointestinal tract and nasal area. A combination of Lactobacillus with Atopobium may be similarly effective for some individuals. From the standpoint of physical exclusion, the attachment of Lactobacillus acts as a block to uropathogens by preventing access to receptor sites. Although complete exclusion of uropathogens theoretically can occur, the most common finding of the results of the present invention is that there is a reduction in uropathogen numbers compared to lactobacilli. In other words, although some lactobacilli may not completely exclude uropathogens, they are still capable of interfering with uropathogen colonization in vivo. Coaggregation is an important element as it allows lactobacilli to form a urogenital mixed flora present in healthy patients. This mixed flora is preferably dominated by lactobacilli and other indigenous gram positive bacteria. It is hypothesized that the lactobacilli of the present invention and some uropathogens coaggregate (Reid et al. 1988, Can. J. Microbiol. 34:344-351, the entire contents of which are incorporated herein by reference), in a way that interferes with the pathogenic process.

The compositions of the present invention may include a growth factor for facilitating the growth of lactic acid bacteria. The phrase “a growth factor for facilitating the growth of lactic acid bacteria,” as used herein is meant a nutrient source or media which supplies a necessary source of food and/or energy for facilitating the growth of lactic acid producing bacteria The growth factor is preferably selective for establishing and maintaining the growth of lactic acid bacteria, preferably Lactobacillus and/or Bifidobacterium, without facilitating extreme growth of pathogenic bacteria The various nutritional requirements essential for bacterial and/or colony growth are normally met when the growth factor contain fermentable carbohydrate, peptone, meat and yeast extract. Supplementations with tomato juice, manganese, acetate and oleic acid esters, especially Tween 80, are stimulatory or even essential for most species and are, therefore, included in most MRS medium. Lactic acid bacteria adapted to very particular substrates may require special growth factors.

Examples of suitable growth factors include, but are not limited to, yeast extracts; gangliosides; salicin; mono-, di- and polysaccharide sugars such as glycogen, glucose, fructose, rharnnose, lactulose, methyl-a-D-mannoside, p-nitrophenol-cc-D-mannoside, maltose, maltodextrin, dextrin, dextran, levan, sialic acid and acetylglucosamine as well as oligosaccharides such as, but not limited to, fructooligosaccharides, galactooligosaccharides and soybean oligosaccharides. Fiber or fermentable substrates such as psyllium may be used in the present compositions as may gums such as guar gum and xanthum gum. Similarly, proteinacious materials such as, peptone, keratin; vegetable; soy and unsaturated fatty acids such as lauric acid and teichoic acids such as lipoteichoic acid and esters such as glycerophosphates or P-glycerophosphates are also useful as growth factors. The growth factor is preferably selected for establishing and maintaining the growth of lactic acid bacteria, most preferably Lactobacillus and/or Bifidobacterium species. Growth factors preferable for use in the compositions of the present invention include lactose, lactulose, rhamnose, oligosaccharides and glycogen. Mixtures of these 15 nutrients may also be used.

More preferably the growth factor of the present invention is an oligosaccharide such as, but not limited to, galactooligosaccharides, soybean oligosaccharides and fructooligosaccharides. Oligosaccharides possess bioadhesive properties which help fix the location of these growth factors for easier access by lactic acid bacteria. Most preferred for use herein are fructooligosaccharides. Lactic acid bacteria, such as Lactobacillus and Bifidobacterium, partially utilize fructooligosaccharides as an energy source by converting it, via fermentation, to lactic acid or a mixture of lactic acid, acetic acid, and CO₂. The lactic acid and other fatty acids produced by this carbohydrate fermentation contribute to the maintenance of low pH which is an important control mechanism for preventing colonization of pathogens.

Chemically, oligofi-uctose is the oligosaccharide fraction of inulin. It is composed of the GFn and Fn type [G=glucose; F=fructose; n=number of fi-utose moieties linked by 0 (2, 1) linkages in a ratio of about 2:1, with n=2-6, and an average degree of polymerization of 4. Inulin is prepared by hot water extraction of chicory roots and is composed of molecules of the GFn type, n ranging as high as 60 with an average degree of polymerization of 10. Fructooligosaccharides suitable for use herein may or may not have non-fiructosyl units in place of fructosyl end units. The same is true for other oligosaccharides with respect to their osyl end units. Non-fructosyl units may include, but are not limited to, polyalcohols such as xylitol, mannitol, and sorbitol.

Fructooligosacchafides most preferred for use in the present Compositions are inulin or oligofructose. Mixtures of these nutrients may also be used.

The present invention may also be useful in maintaining and restoring normal flora of the gastrointestinal tract, nasal passages and urogenital region of men and women and help treat or prevent gastrointestinal upsets including halitosis, and reduce the risk of infection associated with nasal packings.

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A composition for maintaining and restoring normal indigenous urogenital flora in a human comprising one or more bacteria selected from the group consisting of Lactobacillus iners, all clones with at least 97% sequence similarity to Lactobacillus iners, Atopobium spp, and all clones with at least 90% sequence similarity to Atopobium spp as determined by sequences from 16S rRNA genes.

2. The composition according to claim 1 wherein the species of Atopobium spp is Atopobium vaginae and all clones with at least 97% sequence similarity to Atopobium vaginae.

3. The composition of claim 1, wherein said composition further comprises Lactobacillus crispatus.

4. The composition of claim 1, wherein said composition further comprises one or more bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus plantarum, Lactobacillus fermentum, Lactobacillus jensenii, Lactobacillus crispatus, Lactobacillus vaginalis, Lactobacillus mucosae, Lactobacillus paracasei, Lactobacillus rhamnosus, Lactobacillus coleohominis Lactobacillus vaginas, Anaerococcus spp., Clostridium spp., Dialister spp., Enterococcus faecalis, Finegoldia magna, Gardnerella vaginalis, Gemella palaticanis, Lachnospiraceae spp., Leptotrichia spp., Megasphaera spp., Streptococcus spp., Hydrogenophaga palleronii, Comamonas spp., Peptostreptococcus, spp., Aerococcus, spp., Veillonella, spp., Mycoplasma spp., Micromonas spp., and Gemella palaticanis.

5. The composition according to claim 1 may be administered as a suppository, douche, mouth wash, oral tablet, capsule, drink, gum, nasal spray, pad, liner, interlabial device, wipe, pessary, tampon or nasal packing.

6. The composition according to claim 1 wherein said composition is in the form selected from the group consisting of a cream, paste, gum, a suppository, mucoadhesive, liquid dental transport medium, microspheres, an ointment, an oral tablet, a liquid, and a gel.

7. The compositions of claim 1 further comprises a growth factor.

8. A method for maintaining normal indigenous flora to a human's urogenital tract comprising administering a safe and effective amount of one or more bacteria selected from the group consisting of Lactobacillus iners, all clones with at least 97% sequence similarity to Lactobacillus iners, Atopobium spp, and all clones with at least 90% sequence simlarity to Atopobium spp as determined by sequences from 16S rRNA genes.

9. The method of claim 8 wherein each bacteria is administered in a dose of from about 10³ to about 10¹³ cfu/ml.

10. The method of claim 8 wherein each bacteria is administered in a dose of from about 10⁵ to about 10¹⁰ cfu/ml.

11. The method according to claim 8, wherein said composition further comprises Lactobacillus crispatus.

12. The method according to claim 8, wherein said composition further comprises one or more species of bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus. plantarum, Lactobacillus fermentum, Lactobacillus jensenii Lactobacillus crispatus, Lactobacillus vaginalis, Lactobacillus mucosae, Lactobacillus paracasei, Lactobacillus rhamnosus, Lactobacillus coleohominis, Lactobacillus vaginas, Anaerococcus spp., Clostridium spp., Dialister spp., Enterococcus faecalis, Finegoldia magna, Gardnerella vaginalis, Gemella palaticanis, Lachnospiraceae spp., Leptotrichia spp., Megasphaera spp., Streptococcus spp., Hydrogenophaga palleronii, Comamonas spp., Peptostreptococcus, spp., Aerococcus, spp., Veillonella, spp, Mycoplasma spp., Micromonas spp., and Gemella palaticanis.

13. The method of claim 8, wherein said composition is applied directly to the urogenital region of a human with a device selecting from the group consisting of tampons, pantiliners, sanitary pad, interlabial pad, wipes, and pessaries.

14. A method for restoring normal indigenous flora to a human's urogenital tract comprises administering a safe and effective amount of an antibiotic and subsequently administering a safe and effective amount of a composition comprising one or more bacteria selected from the group consisting of Lactobacillus iners, all clones with at least 97% sequence similarity to Lactobacillus iners, Atopobium spp, and all clones with at least 90% sequence similarity to Atopobium spp as determined by sequences from 16S rRNA genes.

15. The method of claim 14 wherein each bacteria is administered in a dose of from about 10³ to about 10¹³ cfu.

16. The method according to claim 14 wherein the antimicrobial is selected from the group consisting of Penicillins, Cephalosporins, Clindamycin, Vancomycin, Tetracyclines, Chlorampheicol, Trimethoprim-sulfamethoxazole, Erythromycins, Glycopeptides, Metronidazole, Sulfa drugs, Iodine and chlorhexidine and mixtures thereof.

17. The method according to claim 14, wherein said composition further comprises Lactobacillus crispatus.

18. The method according to claim 14, wherein said composition further comprises one or more species of bacteria selected from the group consisting of Lactobacillus casei, Lactobacillus gasseri, Lactobacillus fermentum, Lactobacillus amylolyticus, Lactobacillus acidophilus, Lactobacillus casei subs. pseudoplantarum, Lactobacillus brevis, Lactobacillus salivarius, Lactobacillus acidophilus, Lactobacillus plantarum, Lactobacillus fermentum Lactobacillus jensenii. Lactobacillus crispatus, Lactobacillus vaginalis, Lactobacillus mucosae, Lactobacillus paracasei, Lactobacillus rhamnosus, Lactobacillus coleohominis, Lactobacillus vaginas, Anaerococcus spp., Clostridium spp., Dialister spp., Enterococcus faecalis, Finegoldia magna, Gardnerella vaginalis, Gemella palaticanis, Lachnospiraceae spp., Leptotrichia spp., Megasphaera spp., Streptococcus spp., Hydrogenophaga palleronii, Comamonas spp., Peptostreptococcus, spp., Aerococcus, spp., Veillonella, spp, and. Mycoplasma spp., Micromonas spp., and Gemella palaticanis.

19. The method of claim 14, wherein said composition is applied directly to the urogenital region of a human with a device selecting from the group consisting of tampons, pantiliners, sanitary pad, interlabial pad, wipes and pessaries.

20. A composition for maintaining and restoring normal indigenous urogenital flora in a human comprising Lactobacillus iners and all clones with at least 97% sequence similarity to Lactobacillus iners.
